SEQUENCE LISTING (4 sequences)

SEQ ID NO: 1 - DNA, 885 bp, Petunia sp.
atgggaagacaaccttgttgtgacaaacttggagtgaagaaaggtccatggacagctgaagaagacaagaaactcataagttttattcttacaaatggccaatgttgttggcgtgctgttcctaaacttgctggtcttaaacgttgtggtaagagttgtcgactcagatggactaattatcttcgacctgatttgaaaagaggccttcttagtgatgctgaggagaaactggttattgatctccattctcgtcttggaaacaggtggtccaagattgctgcaagattaccaggaaggactgataatgagattaaaaatcattggaacacacatattaagaaaaagcttctcaaaatggggattgatcctgttacacatgaaccactcaagaaagaagcaaatctaagtgatcagcctactacagaatctgatcaaaataaagaaaatggtcatcagcaggtacaagttgtaccacagagtacaaatgtcacagcggcagcagctacctccacagaatttgataataactcttccttctcttcttcagcttcttcgtccgaaaactcttcatgcaccactgatgaatccaagcttgtctttgataaccttagtgaaaatgatccactactaagctgcctactggaagctgatactcctcttatcgattcgccatgggaatttccaatgtcttctactactactgttgaagaaccaaaaagtttcgatagcattatcagtaacatgacatcctgggaagacactttcaattggctttcgggttatcaagaatttggtatcaatgactttggttttgataattgcttcaaccatgtcgaattggacatcttcaaaaccatagataacgtagagaacaggcacggataa

SEQ ID NO: 2 - protein, 294 aa, Petunia sp.
MGRQPCCDKLGVKKGPWTAEEDKKLISFILTNGQCCWRAVPKLAGLKRCGKSCRLRWTNYLRPDLKRGLLSDAEEKLVIDLHSRLGNRWSKIAARLPGRTDNEIKNHWNTHIKKKLLKMGIDPVTHEPLKKEANLSDQPTTESDQNKENGHQQVQVVPQSTNVTAAAATSTEFDNNSSFSSSASSSENSSCTTDESKLVFDNLSENDPLLSCLLEADTPLIDSPWEFPMSSTTTVEEPKSFDSIISNMTSWEDTFNWLSGYQEFGINDFGFDNCFNHVELDIFKTIDNVENRHG

SEQ ID NO: 3 - DNA, 32 bp, Artificial
  Synthetic forward primer designed to amplify the region from nucleotide 573 to 876 of the ODO1 open reading frame for RNAi silencing
aaaaagcaggctcaccactgatgaatccaagc

SEQ ID NO: 4 - DNA, 30 bp, Artificial
  Synthetic reverse primer designed to amplify the region from nucleotide 573 to 876 of the ODO1 open reading frame for RNAi silencing.
agaaagctgggtcctgttctctacgttatc

(19) United States
(12) Patent Application Publication
(10) Pub. No.: US 2008/0127368 A1
(43) Pub. Date: May 29, 2008

(54) Novel Regulatory Protein

(76) Inventors: Michel Albertus Haring, Haarlem (NL); Robert Cornelis Schuurink, Leiden (NL); Julian Cornelis Verdonk, Amsterdam (NL); Arjen J. VanTunen, Wageningen (NL)

Correspondence Address: MORGAN LEWIS & BOCKIUS LLP, 1111 PENNSYLVANIA AVENUE NW, WASHINGTON, DC, 20004, US

(21) Appl. No.: 11/661,758

(22) PCT Filed: Sep. 1, 2005
(86) PCT No.: PCT/NL05/00634

§ 371 Date: Dec. 7, 2007

(30) Foreign Application Priority Data
Sep. 2, 2004 (NL) PCT/NL04/00611

(51) Int. Cl. C12N 15/82; C12P 21/04; C07K 16/18; C07K 14/00; C12N 15/11; C12N 15/00; C12Q 1/68; C12N 5/06; C12N 5/04; C12N 1/20; C12N 1/00; A01H 5/00

(52) U.S. Cl. 800/279; 530/350; 536/23.1; 435/320.1; 435/325; 435/410; 435/252.3; 435/254.11; 800/298; 530/387.5; 435/69.1; 800/278; 435/6

(57) ABSTRACT

The present invention relates to a polypeptide which belongs to the R3R3-type MYB family and which regulates the shikimate pathway towards the production of benzenoids. The shikimate pathway is a biosynthesis pathway through which the three essential aromatic amino acids tyrosine, phenylalanine and tryptophan are synthesized in plants, bacteria and fungi. The present invention provides for the first time a regulatory protein in the shikimate pathway and a means to regulate the biosynthesis of these three essential amino acids which cannot be produced by mammals. At the same time, it opens up the way for the regulation of the biosynthesis of aromatic and non-aromatic compounds which are derived from these essential amino acids. A polypeptide or polynucleotide of the invention may be used in a method for manipulating the transcript levels of the genes of the shikimate pathway towards benzenoids for instance the genes of 3-deoxy-D-arabino-heptulosonate-7-phosphate synthase (DAHPS), 5-enol-pyruvylshikimate-3-phosphate synthase (EPSPS), L-phenylalanine ammonia-lyase (PAL) and chorismate mutase (CM).

### FIELD OF THE INVENTION

The present invention relates to myb regulatory proteins in plants. More in particular it relates to a myb protein of the R2R3 type, the gene which encodes the protein and to applications of this protein and the gene which encodes it.

### BACKGROUND OF THE INVENTION

Scent in plants is an important trait for a number of reasons. For instance production of volatile compounds by the flower can play an important role in the attraction of pollinating insects in the process of reproduction and for a successful and high yield seed set. Alternatively, plants can also produce volatile compounds in their reproductive or vegetative parts that attract pest insects or their predators. In this process the volatile profile determines the sensitivity or resistance against harmful organisms such as pest insects, nematodes or fungi. Interfering and modifying volatile synthesis and release can be an interesting avenue to interfere with plant/insect relations and thereby improve reproductive processes or resistances against pest insects. Scent is also an important trait in the horticulture industry. In many cases scent is not or no longer present due to a broad negligence of this trait in breeding programs. A good example of this is rose, where we see that in most cultivars scent has been lost. There is a tendency and a general interest to (re)introduce scent into commercial ornamental varieties. This can be achieved by traditional breeding. However this requires a lot of time and efforts. Similarly, the taste of vegetable crops like tomato is influenced by the concentration of volatiles. Again, altering taste of tomatoes by traditional breeding is time-consuming.

Volatiles are also important for the Flavour and Fragrance, Food and Cosmetics industries that, in a number of cases, produce natural volatile compounds from flowers, herbs, fruits and spices as a source of flavour and fragrance ingredients for usages in perfumes, foods, cosmetics and so on.

An important class of volatile compounds from plants are the so called benzenoids. These phenolic compounds have a basic C6 skeleton and are produced from the shikimate pathway often in specific organs or specific cell types. A number of plants like orchids and petunia's have flowers that produce benzenoids as the main part of their fragrances. In a number of Petunia hybrida lines benzenoid volatile compounds are specifically released starting at the end of the afternoon and during the night by the petals in a day/night rhythm. To date only limited knowledge is present about the molecular and genetic processes that are involved in this pathway. Only a few of the structural genes have been cloned and characterized and to date no regulatory genes have been identified. It is therefore still largely unknown how plants regulate benzenoids biosynthesis and release.

It would be a great advantage if a reliable way to reproducibly, efficiently and cost effectively block, enhance or modify scent biosynthesis could be achieved in plants or other (micro) organisms.

### SHORT DESCRIPTION OF THE FIGURES

FIG. 1. Quantified emission of volatile benzenoids by Mitchell (W115). Four RNAi lines that show reduced emission of volatiles (TA-1, TA-3, TA-12 and TA-35) and one RNAi line (40) that shows no reduction in benzenoid emission.

FIG. 2. RNA gel blot analysis of Mitchell (M) and RNAi lines 1, 3, 12, 35 and 40 for ODO1 and genes from the shikimate pathway, the synthesis of phenylalanine and t-cinnamic acid, such as DAHP synthase (DAHPS), EPSP synthase (EPSPS), chorismate mutase (CM) and two phenylalanine ammonia lyase genes (PAL1 and 2); for benzylbenzoate transferase (BEBT) and benzoic acid/salicylic acid methyltransferase (BSMT). Transcript levels of floral binding protein 1 (FBP1) are shown to indicate the specificity of ODO1 suppression in the RNAi lines.

FIG. 3 Alignment of the R2R3 domain of ODO1 homologs revealing conserved (dark shades) residues that are indicative of functional conservation.

### DETAILED DESCRIPTION

The present invention relates to a polypeptide with DNA binding activity which has the polypeptide sequence as shown in SEQ ID No. 1, or a variant or derivative thereof.

These polypeptides of the invention belong to the R2R3-type MYB family and regulate the shikimate pathway. The shikimate is the pathway through which the three aromatic amino acids tyrosine, phenylalanine and tryptophane are synthesized. From these compounds other aromatic compounds may be formed. This is the first time that a regulatory protein in the shikimate pathway towards benzenoids has been identified. Therefore, one of the advantages of the present invention is that it provides for the first time a regulatory protein in the shikimate pathway and a means to regulate the biosynthesis of three essential amino acids which cannot be produced by mammals. At the same time, it opens up the way for the regulation of the biosynthesis of aromatic and non-aromatic compounds which are derived from these essential amino acids. Examples of the most important compounds which are derived from the three aromatic amino acids include compounds such as cinnamic acid, coumaric acid, caffeic acid, ferulic acid; compounds from the shikimate pathway, which themselves can be intermediates for many other industrially interesting compounds. To name a few, benzenoids including methylbenzoate, methylsalicylate, benzaldehyde, benzylacetate, benzylbenzoate, vanillin, isoeugenol, and phenyl propanoids including flavonols and anthocyanins. Since these compounds are involved in both primary and secondary metabolism, the skilled person will understand that the protein of the invention provides a means to influence many biosynthetic processes. This includes the regulation of chemicals that are involved in the defence against pathogens, being mostly of phenolic origin, and the regulation of volatile benzenoid emission. Since the shikimate pathway is also present in certain bacteria and fungi, the teaching of the present invention also extends to such systems.

### Variants and Derivatives of Polypeptides of the Invention

As used herein, a “variant” or “derivative” includes a peptide or a non-peptide compound which differs from the recited polypeptide in a substitution, deletion, addition of or fusion with one or more amino acids while retaining the properties of the recited polypeptide. These terms also include peptide or non-peptide compounds which differ from the recited polypeptide in that some glycosylation sites have been introduced or modified while retaining the properties of the recited polypeptide. These terms also include peptide or non-peptide compounds which differ from the recited polypeptide in that modifying groups have been coupled to the peptidic structure, be it covalently or non-covalently, while retaining the properties of the recited polypeptide. In particular, variant and derivatives have retained the capacity to manipulate the transcript levels of the genes of the shikimate pathway towards benzenoids, including the transcript levels of the genes for 3-deoxy-D-arabino-heptulosonate-7-phosphate synthase (DAHPS), 5-enol-pyruvylshikimate-3-phosphate synthase (EPSPS), chorismate mutase (CM) and L-phenylalanine ammonia-lyase (PAL).

In one embodiment, the variant or derivative comprises an amino acid sequence which shows at least 50%, 55%, 60%, 65%, 70%, 75%, preferably 80%, 85%, 90%, 95%, 97%, 98% or 99% identity to the amino acid sequence of SEQ ID NO. 1.

In yet another embodiment, the variant or derivative comprises an amino acid sequence which shows at least 90%, 95%, 97%, 98% or 99% identity to amino acids 13-116 of SEQ ID No. 1. This region of amino acids corresponds to the DNA binding domain of polypeptides of the invention, which is a myb DNA binding domain of the R₂R₃ type. For more information on this type of myb domain, see Stracke et al. (2001) Current Opinion in Plant Biology 4: 447-456.

In yet another embodiment, the variant or derivative comprises an amino acid sequence which show at least 70%, 75% or 80% identity to the region from amino acid 128 to amino acid 294 of SEQ ID No. 1. Preferably, the variant or derivative comprises an amino acid sequence which shows at least 85%, 87%, 89% or 90% identity to the region from amino acid 128 to amino acid 294 of SEQ ID No. 2. Most preferably, the variant or derivative comprises an amino acid sequence which shows at least 94%, 97%, 98% or 99% identity to the region from amino acid 128 to amino acid 294 of SEQ ID No. 1.

In yet another embodiment, the variant or derivative is a polypeptide which differs from the recited polypeptide only in conservative substitutions. As used herein, a “conservative substitution of an amino acid” refers to the substitution of one amino acid for another that has similar properties. For instance, when an amino acid with hydrophobic properties is replaced by another amino acid with hydrophobic properties.

The terms peptide and polypeptide are used essentially interchangeably herein to refer to a molecule which contains a string of amino acids.

Amino acid identity may be readily calculated by known methods, including but not limited to those described in (Computational Molecular Biology, Lesk, A. M., ed., Oxford University Press, New York, 1988; Biocomputing: Infomatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988). Preferred methods to determine identity are designed to give the largest match between the sequences tested. Methods to determine identity are codified in publicly available computer programs. Preferred computer program methods to determine identity and similarity between two sequences include, but are not limited to, the GCG program package (Devereux, J., et al., Nucleic Acids Research 12 (1):387 (1984)), BestFit, BLASTP, BLASTN, and FASTA (Altschul, S. F. et al., J. Mol. Biol. 215:403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215:403-410 (1990). The well-known Smith Waterman algorithm may also be used to determine identity. Preferred parameters for polypeptide sequence comparison include the following: 1) Algorithm: Needleman and Wunsch, J. Mol, Biol. 48:443-453 (1970) Comparison matrix: BLOSSUM62 from Hentikoff and Hentikoff, Proc. Natl. Acad. Sci. USA. 89:10915-10919 (1992); Gap Penalty: 12; and Gap Length Penalty: 4. A program useful with these parameters is publicly available as the “Ogap” program from Genetics Computer Group, located in Madison, Wis. The aforementioned parameters are the default parameters for peptide comparisons (along with no penalty for end gaps).

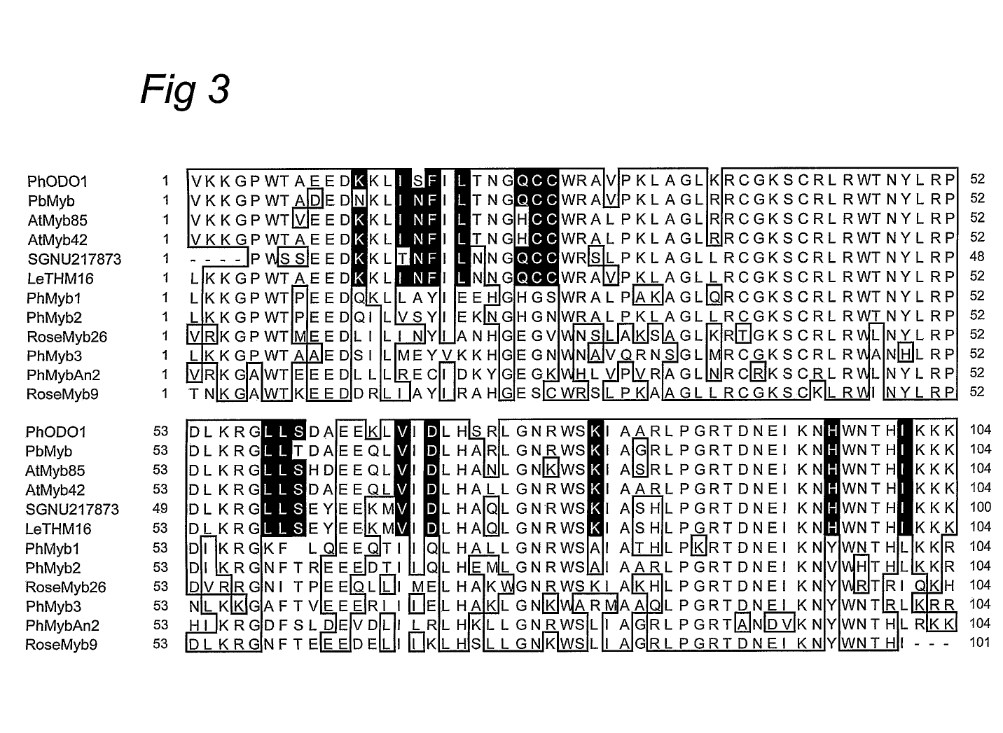

For instance, tomato ESTs SGNU217873 and LeHTM16 (see FIG. 3) are homologues of ODO1. Both cDNAs share homology with the ODO1 family proteins PbMYB, AtMYB42 and AtMYB85 in the R2R3 domain at characteristic positions. This homology is indicative of functional conservation. Although tomato produces no significant amounts of benzenoids in its flowers, several benzenoids accumulate in ripe tomato fruit. ESTs of SGNU217873 have been found only in flower buds and ovaries (see SGN transcript database, http://www.sgn.cornell.edu/) and expression increases during ripening indicating that this gene is involved in benzenoid production during fruit ripening. LeHTM16 is not induced during fruit ripening, therefore this MYB will regulate processes elsewhere in the plant.

### Polynucleotides of the Invention

In another aspect, the present invention provides an isolated, recombinant or synthetic polynucleotide comprising a nucleotide sequence with a sequence as shown in SEQ ID. No. 2, or a variant thereof which shows at least 50%, 55%, 60%, 65%, 70%, 75%, preferably 80%, 85%, 90%, 95%, 97%, 98% or 99% identity to SEQ ID. No. 2 and which encodes a protein with regulatory activity for the shikimate pathway towards benzenoids.

In one embodiment, a polynucleotide of the invention comprises a polynucleotide sequence which encodes a polypeptide with an amino acid sequence as shown in SEQ ID NO. 1, or a fragment of thereof with regulatory activity for the shikimate pathway towards benzenoids.

Also encompassed by the present invention are polynucleotide sequences which have a sequence which is complementary to the polynucleotide sequence of SEQ ID No. 2, such as anti-sense RNA or other inhibitory RNA, e.g. such as used in RNAi; or which hybridise under stringent conditions to part of the sequence of SEQ ID NO. 2. These complementary and hybridising sequences may be of any length and the skilled person will understand that the appropriate length should be adapted to the purpose for which the sequence is to be used. For instance, for post-transcriptional silencing double stranded RNA of any length may be used in plants.

Identity of two nucleotide sequences is determined using the methods mentioned above. The terms nucleotide sequence, nucleic acid and polynucleotide are used essentially interchangeably in this application to refer to a sequence of nucleotides. The polynucleotides of this invention may include genomic sequences, extra-genomic and plasmid-encoded sequences and smaller engineered gene segments that express, or may be adapted to express, proteins, polypeptides, peptides and the like. Polynucleotides of the invention may be single-stranded (coding or antisense) or double-stranded, and may be DNA (genomic, cDNA) or RNA molecules. They may be isolated, recombinant or synthetic. RNA molecules may include heterogeneous nuclear RNA (hn RNA) molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials.

“Isolated,” as used herein, means that a polynucleotide is substantially free from other nucleic acid sequences, and that the polynucleotide does not contain large portions of unrelated sequences, such as large chromosomal fragments or other functional genes or polypeptide coding regions. Of course, this refers to the polynucleotide molecule as originally isolated, and does not exclude genes or coding regions later added to the segment by the hand of man.

### Vectors of the Invention

In another aspect, the invention relates to a vector comprising a polynucleotide of the invention. Vectors which advantageously may be used include well-known plant vectors such as pK7GWIG2(I) and pgreen, as well as state of the art vectors used for transforming and expressing proteins in microorganisms. See also Arabidopsis, A laboratory manual Eds. Weigel & Glazebrook, Cold Spring Harbor Lab Press (2002) and Maniatis et al. Molecular Cloning, Cold Spring Harbor Lab (1982).

### Host Cells of the Invention

In yet another aspect, the invention relates to a host cell comprising a polynucleotide or a vector according to the invention. Suitable host cells according to the invention include plant cells, yeast cells, fungal cells, algal cells, human cells and animal cells. Examples of suitable plant cells include tomato and Arabidopsis. Examples of suitable yeast cells include Saccharomyces cerevisiae and Pichia pastoris. Examples of suitable fungal cells include Aspergillus. Examples of suitable animal cells include insect cells, e.g. from Spodoptera frugiperda; mammalian cells such as Chinese hamster ovary cells or PERC6 cells. A variety of state of the art cell lines may be used, such as the Flp-In cell lines (Invitrogen). As indicated above, a variety of vectors for introducing a polynucleotide of the invention into the host cell may be used. These vectors may be cloning vectors, expression vectors, silencing vectors which may be chosen from, for example, plasmid DNA vectors, viral DNA vectors (such as adenovirus or adeno-associated vectors), viral RNA vectors (such as retroviral) or viral plant vectors, such as tobacco rattle virus and potato virus X.

Also the production of the polypeptide of the invention by cell free extract is encompassed by the present invention. Methods for production in cell free extracts are known in the art. See for example Pelman and Jackson (1976) Eur. J. Biochem 67: 247-56.

Host cells of the invention may be used to produce polypeptides of the invention. This involves culturing a host cell according to the invention under conditions which allow for the production of the polypeptide, and, optionally, recovering the polypeptide. In a preferred embodiment, a recombinant polypeptide with DNA binding activity is produced.

In one embodiment, the host cell is a transgenic plant in which the gene which encodes a protein according to the invention is silenced. As a result, enzymes in the shikimate pathway towards benzenoid production will be down regulated, for instance the transcript levels of the genes for 3-deoxy-D-arabino-heptulosonate-7-phosphate synthase (DAHPS), 5-enol-pyruvylshikimate-3-phosphate synthase (EPSPS), chorismate mutase (CM) and L-phenylalanine ammonia-lyase (PAL) will be reduced, and the volatile profile of the plant will be changed.

In another embodiment, the host cell is a transgenic plant in which the gene encoding the protein of the invention is over expressed and the enzymes of the shikimate pathway are upregulated, for instance the transcript levels of the genes for 3-deoxy-D-arabino-heptulosonate-7-phosphate synthase (DAHPS), 5-enol-pyruvylshikimate-3-phosphate synthase (EPSPS), chorismate mutase (CM) and L-phenylalanine ammonia-lyase (PAL) are increased and the volatile profile is modified in such a way that more scent is produced. A transgenic plant with increased levels of EPSPS production is of particular interest in chemical defence strategies. In particular in those strategies were plant protection products are used which contain glyphosate, because a plant with increased levels of EPSPS will have increased resistance towards glyphosate.

In yet another embodiment, the host cell is a transgenic plant in which the gene encoding the protein of the invention is overexpressed and the volatile profile is modified in such a way that it strengthens the plant's chemical defence system towards pathogenic organisms by increasing the benzenoid production. This includes benzenoids which function as, insecticide, fungicide, nematicide, molluscicide or rodenticide.

In another embodiment, the host cell is a transgenic plant cell resulting in a transgenic plant in which the gene encoding the protein of the invention is overexpressed so that co suppression occurs. As a result, the gene will be silenced (Jorgensen et al. (1996) Plant Mol Biol 31: 957-973) and transcript levels of the genes of the shikimate pathway towards benzenoid production for DAHPS, EPSPS, CM and of PAL will be reduced.

In another embodiment, the host cell is a transgenic plant cell resulting in a transgenic plant in which the gene encoding the protein of the invention is silenced in other ways known in the art.

In yet another embodiment, the host cell is a transgenic plant cell resulting in a transgenic plant in which the gene encoding the protein of the invention is silenced and the volatile profile is modified in such a way that pest insects are not or less attracted. In yet another embodiment, the host cell is a transgenic plant cell resulting in a transgenic plant in which the gene encoding the protein of the invention is introduced in the host which did not contain the gene, or not in active form, before introduction. In this way it is possible to regulate the shikimate pathway and thus the biosynthetic pathway of aromatic compounds.

### Antibodies

Antibodies directed against polypeptides of the invention are also encompassed in the present invention. Methods for generating polyclonal and monoclonal antibodies are generally known in the art (see e.g. Harlow and Lane (1988) Antibodies, A Laboratory Manual, Cold Spring Harbor Laboratory, Cold Spring Harbor Laboratory Press, New York). The antibody may be used as such, but preferably the antibody is labeled with a detectable label. Suitable antibody labels are known to the person skilled in the art and include, but are not limited to, radioactive labels, electron dense labels, enzymatic labels and fluorescent labels. In a preferred embodiment, enzymatic or fluorescent markers are used, such as alkaline phosphatase, horse radish peroxidase and fluorescein.

Also intracellular produced antibodies, so-called intrabodies are encompassed by the present invention. The construction of intrabodies has been described in the art, e.g. in U.S. Pat. No. 6,004,940 and in WO 01/48017.

Antibodies of the invention may be used in a method for identifying or detecting scenting flowers. The method comprises contacting plant material with an antibody of the invention; followed by detection whether or not binding to a polypeptide of the invention has taken place. Such method is also encompassed by the present invention. The protein may be recovered using recovery techniques known in the art, e.g. as described in Methods Enzymol. vol. 182, Guide to protein purification. Eds. M. P. Deutscher (1990) Academic Press Inc.

### Methods of the Invention

A polypeptide, a polynucleotide, a vector or an antibody or fragment thereof according to the invention (collectively called compounds of the invention) may be used in a method for manipulating the transcription levels of the genes of the shikimate pathway, for instance the transcript levels of the genes for 3-deoxy-D-arabino-heptulosonate-7-phosphate synthase (DAHPS), 5-enol-pyruvylshikimate-3-phosphate synthase (EPSPS), chorismate mutase (CM) and L-phenylalanine ammonia-lyase (PAL). Through these enzymes, downstream biosynthetic processes may be influenced. For instance, compounds of the invention may be used in a method to regulate scent in flowers or to regulate resistance to pest insects or pathogenic organisms. Therefore, the use of a polypeptide, a polynucleotide, a vector or an antibody or fragment thereof according to the invention for modifying the profile of volatile scent compounds in plants; for regulating the transcription levels of genes from the shikimate-phenylalanine synthesis pathway; for regulating the transcription levels of genes from the phenylpropanoid pathway; for regulating the transcription levels of genes involved in benzenoid biosynthesis; or for regulating the biosynthesis of aromatic amino acids, in particular the biosynthesis of phenylalanine, tyrosine and tryptophane, is also encompassed in the present invention.

In one embodiment, compounds of the invention are used in a method for producing a plant in which the profile of volatile scent compounds can be modified. The method comprises introducing into a plant a polynucleotide of the invention. In one embodiment, the polynucleotide of the invention is introduced in the genome of the plant.

In yet another embodiment, compounds of the invention are used in a method for discriminating between scenting and non-scenting plants. The method comprises:
contacting plants with a compound according to the invention; and
detecting binding to such compound or detecting polymorfism within nucleotides of the invention.

In yet another embodiment, compounds of the invention are used in genetic analysis or marker assisted selection in plant breeding. In particular, they may suitably be used in marker assisted selection based on PCR, such as (restriction fragment length polymorphism (RFLP), amplified fragment length polymorphism (AFLP), random amplified polymorphic DNA (RAPD), single nucleotide polymorfism (SNP) and microsatellites. For a further description of these techniques, see for example Welsh & McClelland (1990) Nucleic Acids Research 18: 7213-7218; Vos et al. (1995) Nucleic Acids Research 23: 4407-4414 and Struss & Plieske (1998) Theoretical & Applied Genomics 97:308-315. By comparing the restriction pattern or nucleotide sequence differences of two breeding lines or cultivars, polymorphism within the gene which encodes a polypeptide of the invention may be identified. This allows for the very early selection of a trait associated with the polypeptide of the invention, such as scent or increased benzenoid levels.

In yet another embodiment, compounds of the invention are used in a method for increasing resistance to a pest insect by down regulation of a polypeptide according to the invention. Down regulation will change the profile of volatile benzenoids which are emitted and as a result less pest insects will be attracted or predators of pest insects will be attracted.

In yet another embodiment, compounds of the invention are used in a method for increasing resistance to pathogenic organisms by up regulating the expression of the polypeptide according to the invention. Up regulation will lead to a change in the profile of benzenoids which are produced, which are part of the chemical defense mechanism of a plant against pathogenic organisms such as pathogenic bacteria and fungi.

Methods for up and down regulating the expression of a polypeptide in plant and animal systems are known in the art. Upregulation is based on overexpression of the polypeptide of interest in the whole plant or in specific plant parts, such as petals and leaves.

Downregulation may take place at the DNA level, by interfering with e.g. transcription. Alternatively, it may interfere at the RNA level, e.g. by interfering with the translocation of the RNA to the site of protein translation, or with the translation of protein from the RNA, or with the splicing of the RNA to yield one or more mRNA species The overall effect of such interference with expression is a decrease (inhibition) in the expression of the gene. Interference on RNA level is preferred. Suitable ways to achieve interference on RNA level are through RNAi using double stranded or hairpin RNA; through silencing using siRNA; or through cosuppression. See for instance, Hammond & Hannon (2001) Nature Rev Gen 2: 110-119, Arabidopsis, A laboratory manual Eds. D. Weigel & J Glazebrook (2002), CSHL Press and Cogoni & Macino (2000) Genes Dev 10: 638-643.

Downregulation also includes translational and post-translational inhibition. Methods for translational and post-translational inhibition are well-known in the art. Suitably, miRNAs which are endogenous 21-24 nt RNAs that primarily act as repressors of translation and therefore affect only protein expression levels may be used; phosphorylation, acetylation, methylation, glycosylation, prolyl isomerization, sialylation, hydroxylation, oxidation, glutathionylation, and ubiquitination may be used; or antibodies, antibody fragments and chemical and peptide inhibitors may also be used for this purpose.

Methods to identify inhibitors are known and described in the art, and include such methods as screening libraries of peptidomimetics, peptides, DNA or cDNA expression libraries, combinatorial chemistry and, particularly useful, phage display libraries. These libraries may be screened for binding molecules by contacting the libraries with substantially purified polypeptide, fragments thereof or structural analogues thereof. In a preferred embodiment, an inhibitor targets the DNA binding domain of a polypeptide of the invention. As used herein, the term “inhibitor” includes molecules such as peptides, peptide-sequences, peptide-like molecules and non-peptide molecules that bind to a compound of the invention.

### EXAMPLES

### Plant Material and Transformation

Petunia hybrida cv. Mitchell (also referred to as line W115; P. axillaris×(P. axillaris×P. hybrida Rose of Heaven)) and W138 plants were grown as previously described in Verdonk et al. Phytochemistry 62, 997-1008 (2003). Plants bearing at least three mature flowers were used in all experiments. Transgenic Petunias were obtained via Agrobacterium tumefaciens (strain GV3101::pMP90) mediated transformation, by dipping leaf cuttings in bacterial cultures (o/n at 28° C., 10 time diluted). Transgenic calli were selected on MS-medium containing 150 mg/ml kanamycin, from which plants were subsequently regenerated as described in Lucker et al. Plant Journal 27, 315-324 (2001). Rooting plants were tested for the presence of the ntpII gene and of the RNAi construct using PCR. Positive-plants were transferred to the greenhouse.

### Selection and Identification of ODO1

The construction, labelling and analysis of the petal-specific DNA microarrays have been described previously in Verdonk et al. Phytochemistry 62, 997-1008 (2003). Three experiments were compared: Mitchell petals from 9.00 h with those from 15.00 u; petals from 12.00 h with those from 15.00 h and Mitchell petals from 15.00 h with W138 (a non-scenting cultivar) petals from 15.00 h. cDNAs that were significantly (see Verdonk et al. Phytochemistry 62, 997-1008 (2003)) and co-ordinately upregulated with scent emission and that were not upregulated in W138, were sequenced. One of them was identified as a MYB-homologue, but also DAHPS, EPSPS, CM, PAL1 and 2 and BEBT were selected from these microarray experiments. RNA gel blot analysis was performed as described in Verdonk et al. Phytochemistry 62, 997-1008 (2003); specific 3′ UTR probes were used for PAL1 and 2.

### Generation of the RNAi Silencing Construct

Two primers including Gateway™ (Invitrogen life technologies, Carlsbad, Calif., USA) adapters were designed to amplify the region from nucleotide 573 to 876 of the ODO1 open reading frame. Forward primer: 5′-aaa aag cag gct CAC CAC TGA TGA ATC CAA GC-3′; reverse primer: 5′-aga aag ctg ggt CCT GTT CTC TAC GTT ATC-3′ (the lower case letters represent the Gateway™ adapters built in the primers). The amplified PCR product was cloned in the pDONR207 vector and transferred to the RNAi-destination vector pK7GWIWG2(I) (whose nptII gene confers kanamycin resistance to plant cells; VIB, Gent, Belgium), in E. coli DH5α as described by the manufacturer (Invitrogen life technologies). The construct was sequenced and subsequently transformed to A. tumefaciens GV3101::pMP90-cells using standard molecular biological techniques.

### Sampling Volatiles

Volatiles were collected by placing cut flowers in a glass Erlenmeyer with water, which was placed in a 1 litre bottle that was subsequently closed with a lid containing a glass air inlet and outlet. Carbon-filtered air was led in the bottles by applying vacuum on the outlet of the bottle. The headspace of the flowers was collected during 20 h by trapping the outgoing air on 150 mg Tenax TA in 5 mm wide glass tubes, thereby sampling 100% of the volatiles emitted by the flowers. The tenax was eluted with 2 ml pentane:diethylether (4:1) that contained 8.37 ng/μl α-terpinene as internal standard. The volatiles in the eluent were analysed through capillary gas chromatograph-mass spectrometry. One μl of the eluent was injected into an Optic (ATAS, GL, International) injection port at 250° C. The split flow was 0 ml min⁻¹ for 2 minutes and then 25 ml min⁻¹ until the end of the run. Compounds were separated on a capillary DB-5 column (10×180 μm, film thickness 0.18 μm; Hewlett Packard) at 40° C. for 3 min and then to 250° C. at 30° C. min⁻¹ with He as carrier gas. The column flow was 3 ml min⁻¹ for 2 min and 1.5 ml min⁻¹ thereafter. Mass spectra of eluting compounds were generated at 70 eV (ion source at 200° C.) and collected on a Time-of-Flight MS (Leco, Pegasus III, St. Joseph, Mich., USA) with a 90 s acquisition delay at −1597 eV, at an acquisition rate of 20 spectra s⁻¹. Compounds were identified and quantified on the basis of synthetic external standards of known concentration and the internal standard and as previously described in Kant, et al. Plant Physiology 135, 483-495 (2004). Each line was measured at least three times. For each experiment the fresh weight of the flowers was determined.

### Example 1

### Identification and Expression of a Transcription Factor Involved in Floral Scent Regulation in Petunia

To identify components involved in floral scent regulation in Petunia, a targeted transcriptomics approach was used. The transcriptome of flowers that were scenting were compared with that of flowers that were just about to scent and with flowers of Petunia cultivars that do not scent, using a dedicated, highly specific microarray. Transcription factors with increased transcript levels just before scenting and very low transcript levels in non-scenting Petunias were selected. One transcription factor, ODO1 (ODORANT 1), is described in detail here.

Consistent with a role in regulating floral scent, ODO1 transcript levels increased between noon and 14.00 h at the onset of volatile benzenoid emission. Transcript levels of ODO1 increased transiently and were back at their lowest level early the next morning. Expression of ODO1 was restricted to the tube and petals of the flowers. During development of the flower, transcripts of ODO1 were detected just after the flower opened till senescence, after approximately 6 days. Transcript levels of ODO1 were very low in Petunia hybrida line WI 38, a Petunia line which can be considered a non-scenting line.

### Example 2

### Characterisation of the Transcription Factor which is Involved in Floral Scent Regulation

Sequencing of ODO1 revealed that it encodes a putative protein of 294 amino acids (SEQ ID No. 1), with high homology to members of the R2R3-type MYB family, without a nuclear localisation signal. Though the N-terminal R2R3-domain (amino acids 1-128 of SEQ ID No. 1) contains the highly conserved motifs and amino acids presumably involved in DNA-binding to certain variable core motives and formation of a helix-turn-helix structure, the C-terminus has no homologous sequences in the Genbank database. Phylogenetic tree analyses puts ODO1 closest to a MYB from Pimpinella brachicarpa and two from Arabidopsis thaliana AtMYB42 and AtMYB85, of which the functions are unknown. Seventeen of the more variable amino acids of the R2R3-domain are conserved in these three proteins.

### Example 3

### Silencing of the ODO1 Gene in Order to Establish its Role in the Floral Scent Regulation

To investigate the role of ODO1 in floral scent regulation, a transgenic approach was used. The expression in Mitchell of ODO1 was suppressed through RNAi. Since ODO1 is only expressed in the tubes and petals and not in any other tissue, we used a constitutive promoter for the RNAi construct. This promoter drives sequences encoding the C-terminus of ODO1, which showed no homology to other genes in the database, so that it would suppress accumulation of ODO1 transcripts. As a negative control we used the intron of ODO1 for a RNAi construct to transform the line Mitchell as well. Each independent transformant was analysed for ODO1 transcript levels in their flowers at 17.00 h when its transcripts are high in the parental Mitchell line. To rapidly investigate volatile production by individual flowers of each transgenic line, we used a targeted metabolomics approach as described in Verdonk et al. (2003) Phytochemistry 62, 997-1008. Subsequently, the volatile emission of flowers of lines that showed less volatile production were quantified and compared with the volatile emission of the parental line. From these transcript and volatile analyses there appeared to be a clear correlation between ODO1 transcript levels and emission of volatile benzenoids.

### Example 4

### Quantification of Volatile Emissions by Silenced Transgenic Plants and Control Plants

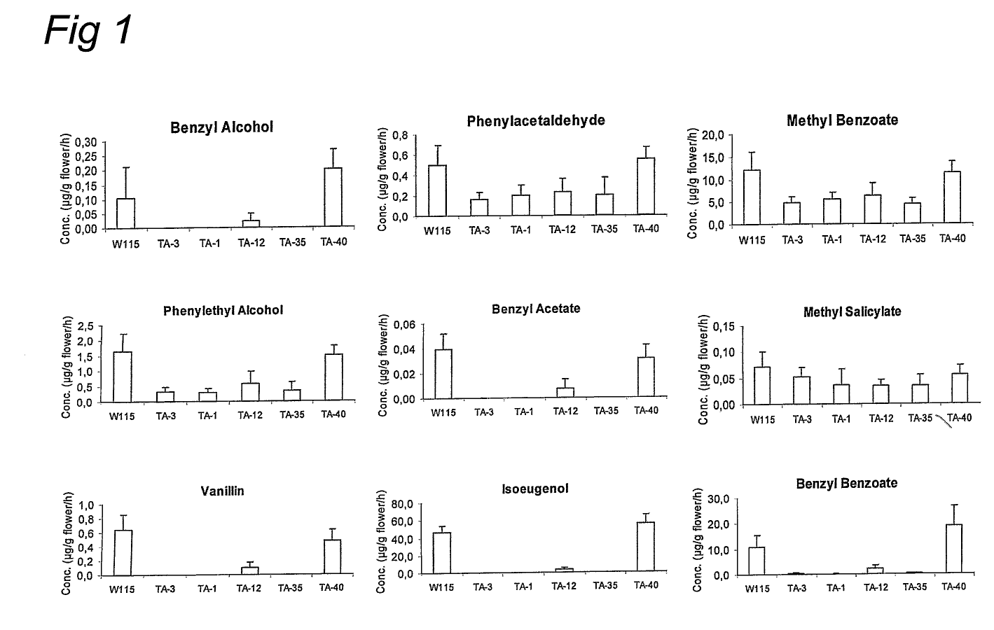

Quantitative analysis of the emitted volatiles for independent transgenic lines is shown in FIG. 1 and revealed that the emission of methylbenzoate, benzylbenzoate and isoeugenol were the most affected volatiles in the transgenic lines. The emission of methylbenzoate was reduced up to 50% and of benzylbenzoate and isoeugenol up to 95%. Vanillin, which is emitted in low amounts by Mitchell, could not be detected in the headspace of RNAi-line 3. The suppression of ODO1 in the lines were this reduction of emission occurred was by far the strongest. The lines that showed no suppression of ODO1 had no reduction of volatile emission. Transcript levels of Floral Binding Protein 1 (FBP1), a protein involved in floral development, were clearly not affected by ODO1, indicating that the ODO1 targets are highly specific.

### Example 5

### The Effect of Silencing of the ODO1 Gene on Transcript Levels of Enzymes in the Shikimate Pathway and of L-Phenylalanine Ammonia-Lyase (PAL)

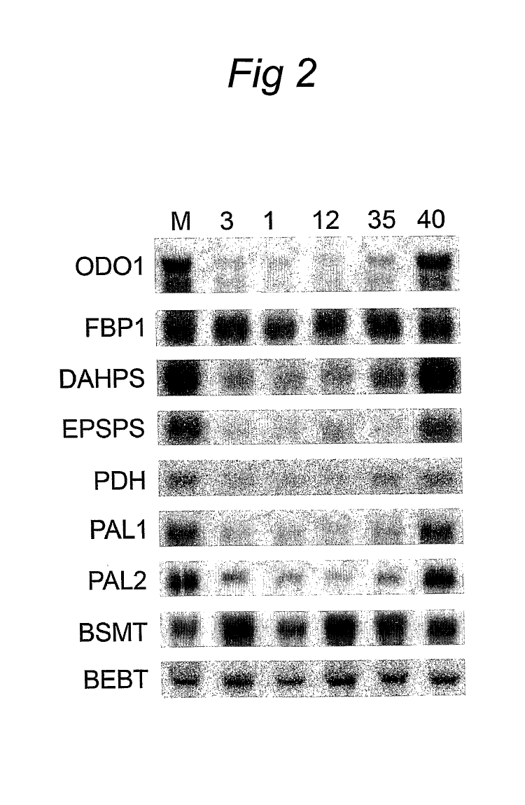

The exact pathways leading to the synthesis of methylbenzoate, benzylbenzoate and isoeugenol are not known, but the first precursor, trans-cinnamic acid, is made by conversion of L-phenylalanine by L-phenylalanine ammonia-lyase (PAL). The shikimate pathway leads to the biosynthesis of L-phenylalanine. To investigate whether ODO1 affected the transcript levels of enzymes in the shikimate pathway and of PAL we performed RNA-gel blot analyses. FIG. 2 shows that transcript levels of the first enzyme in the shikimate pathway, 3-deoxy-D-arabino-heptulosonate-7-phosphate synthase (DAHPS) were much lower in the RNAi plants than in Mitchell. Furthermore, transcript levels of 5-enol-pyruvylshikimate-3-phosphate synthase (EPSPS) and PAL were also reduced in the RNAi plants (FIG. 2). Interestingly, these results clearly show that odo1 not only regulates floral scent, but also regulates enzyme levels earlier in the shikimate pathway.

1. A polypeptide with DNA binding activity which is selected from:
(a) a polypeptide which shows at least 50% identity to the amino acid sequence of SEQ ID No. 1;
(b) a polypeptide which comprises an amino acid sequence which shows at least 90% identity to the region of amino acid 13-116 of SEQ ID No. 1;
(c) a polypeptide which comprises an amino acid sequence which show at least 70% identity to the region from amino acid 128 to amino acid 294 of SEQ ID No. 1.

2. A recombinant or synthetic polynucleotide comprising a polynucleotide selected from the group:
(a) a polynucleotide which is at least 50% identical to the nucleotide sequence as shown in SEQ ID No. 2 or a fragment thereof which encodes a peptide with regulating activity for the shikimate pathway towards benzenoids; and
(b) a polynucleotide which encodes a polypeptide according to claim 1, or a fragment of the polypeptide with regulating activity for the shikimate pathway towards benzenoids;
(c) a polynucleotide which has a sequence which is complementary to the polynucleotide sequence of SEQ ID No. 2;
(d) a polynucleotide sequence which hybridises under stringent conditions to part of the sequence of SEQ ID No. 2.

3. A vector comprising a polynucleotide according to claim 2.

4. A host cell comprising a polynucleotide according to claim 2 or a vector according to claim 3.

5. A host cell according to claim 4 wherein the host cell is a plant cell, a bacterial cell, a yeast cell, a fungal cell or an animal cell.

6. A transgenic plant comprising a polynucleotide according to claim 2.

7. A compound which binds to the polynucleotide of claim 2 or to the polypeptide of claim 1, wherein the compound is preferably an antibody, an antigen binding fragment thereof, or a derivative thereof, or a polynucleotide with a sequence which is complementary to part of the sequence of a polynucleotide according to claim 1.

8. A method for producing a recombinant polypeptide with regulating activity for the shikimate pathway towards benzenoid comprising: —culturing a host cell according to claim 3 or 4 under conditions which allow for the production of the polypeptide and recovering the polypeptide.

9. The use of a polynucleotide according to claim 2, a vector according to claim 3 or a host according to claim 4 or 5, a polypeptide according to claim 1 or a compound according to claim 8 for modifying the profile of volatile scent compounds in plants; for regulating the transcription levels of genes from the shikimate phenylalanine synthesis pathway; for regulating the transcription levels of genes from the phenylpropanoid pathway; for regulating the transcription levels of genes involved in benzenoid biosynthesis; or for regulating the biosynthesis of aromatic aminoacids, in particular the biosynthesis of phenylalanine, tyrosine and tryptophane.

10. A method for producing a plant in which the profile of volatile scent compounds can be modified, which method comprises introducing into a plant genome a polynucleotide according to claim 2 as mentioned under (a) or (b).

11. A method for regulating scent in flowers, which method comprises manipulating the level of expression of a protein encoded by a polynucleotide according to claim 2.

12. A method for discriminating between scenting and non-scenting plants, which method comprises:
contacting plants with a compound according to claim 8; and
detecting binding to a polypeptide according to claim 1 or detecting polymorfism within nucleotides according to claim 2 as mentioned under (a) or (b).

13. A method for regulating or modifying resistance of a plant to a pest insect or pathogenic organism, which method comprises modifying the expression of a polypeptide according to claim 8.

14. A method for regulating in a plant the transcription levels of genes from the shikimate phenylalanine synthesis pathway; for regulating the transcription levels of genes from the phenylpropanoid pathway; for regulating the transcription levels of genes involved in benzenoid biosynthesis; or for regulating the biosynthesis of aromatic aminoacids, in particular the biosynthesis of phenylalanine, tyrosine and tryptophane, which method comprises:
(a) modifying the transcription level of a polynucleotide according to claim 2 as mentioned under (a) or (b);
(b) modify the expression level of a polypeptide according to claim 1; or
(c) introducing a compound according to claim 8 in a plant.

15. Use of a polynucleotide of the invention in a method for genetic analysis or marker assisted selection.

16. Use of a polypeptide according to claim 1 in plant breeding.
